(19) United States
(12) Patent Application Publication
(10) Pub. No.: US 2025/0084133 A1
(43) Pub. Date: Mar. 13, 2025

(54) METHOD FOR RECOVERING PROTEIN FROM SURIMI-RINSING LIQUID BASED ON LOW-TEMPERATURE PLASMA TECHNOLOGY

(71) Applicant: Jiangsu University, Jiangsu (CN)

(72) Inventors: Ruichang GAO, Jiangsu (CN); Xin WANG, Jiangsu (CN); Tong SHI, Jiangsu (CN); Mengzhe LI, Jiangsu (CN); Li YUAN, Jiangsu (CN)

(73) Assignee: Jiangsu University, Jiangsu (CN)

(21) Appl. No.: 18/559,798

(22) PCT Filed: Dec. 30, 2022
(86) PCT No.: PCT/CN2022/143680

§ 371 Date: Nov. 9, 2023

(30) Foreign Application Priority Data
Dec. 13, 2022 (CN) 202211594301.2

(51) Int. Cl. C07K 14/46; C07K 1/36

(52) U.S. Cl. CPC C07K 14/461; C07K 1/36

(57) ABSTRACT

A method for recovering a protein from a surimi-rinsing liquid based on a low-temperature plasma technology is provided. The method includes: collecting fish flesh, and chopping the fish flesh to obtain surimi; adding the surimi to water, mixing for rinsing, centrifuging to collect a supernatant, which is the surimi-rinsing liquid; adjusting an input power of a plasma device to 300 W to 800 W, placing a spray gun of the plasma device below a liquid level of the surimi-rinsing liquid, turning on a power supply, and exciting plasma with air as a gas source, such that the plasma acts evenly on the surimi-rinsing liquid for 2 min to 6 min; and allowing the low-temperature plasma treated surimi-rinsing liquid to stand in an environment with a temperature of 10° C. or lower, centrifuging to collect a precipitate, such that the recovery of the protein from the surimi-rinsing liquid is completed.

### TECHNICAL FIELD

The present disclosure belongs to the technical field of foods, and specifically relates to a method for recovering a protein from a surimi-rinsing liquid based on a low-temperature plasma technology.

### DESCRIPTION OF RELATED ART

In recent years, surimi products have been widely favored and increasingly demanded by consumers due to their characteristics such as high protein contents, low fat contents, and tender tastes. Surimi products are gel-like foods with specified elasticity that are prepared from frozen surimi as a raw material through grinding, shaping, gelation, and the like. Surimi is a product prepared from fish as a raw material through meat separation, rinsing, fine filtration, dehydration, chopping, and freezing. The traditional surimi production processes all require a rinsing treatment, and the rinsing treatment can remove substances such as fishy substances, pigments, fats, and some inorganic salts in fish flesh, thereby improving the gel performance of surimi and increasing the whiteness. However, the rinsing also leads to a loss of nutrients such as water-soluble proteins, fats, and inorganic salts, and an amount of proteins lost due to the rinsing is about one-third of a total amount of water-soluble proteins in fish. In addition, the rinsing involves a large water consumption, and according to statistics, the production of each ton of surimi products leads to about 8 tons of a rinsing waste liquid, and wastewater produced after the rinsing needs to be treated before being discharged, which increases a production cost. The recovery of proteins from a rinsing liquid can reduce a waste of resources, improve a utilization rate of by-products in a surimi production process, reduce the environmental pollution and have specified economic and environmental benefits.

Currently, methods for recovering proteins from a surimi-rinsing liquid mainly include membrane separation methods, flocculation methods, and isoelectric precipitation methods. The membrane separation methods have an excellent recovery effect, but face the problem of membrane contamination. The flocculation methods involve simple operations and a high recovery rate, but a flocculating agent is easy to cause secondary contamination to subsequent processing of recovered proteins, and there is a high use cost. The isoelectric precipitation methods have advantages such as simple operations and low costs, but have a poor recovery effect.

As an emerging non-thermal processing technology, the low-temperature plasma technology has been used in wastewater treatment. The low-temperature plasma technology is considered to be eco-friendly, and only requires a discharge device and air to treat water without side effects to the water. Therefore, the low-temperature plasma technology is a research hotspot in wastewater treatment, but the use of the low-temperature plasma technology in recovery of a protein is rarely reported.

### SUMMARY

In the present disclosure, the feasibility of the method is objectively evaluated according to protein recovery rates for a surimi-rinsing liquid under different low-temperature plasma treatment times.

The method of the present disclosure includes steps such as preparation of a surimi-rinsing liquid, low-temperature plasma treatment of the surimi-rinsing liquid, precipitation and recovery of proteins, and data processing.

A method for recovering a protein from a surimi-rinsing liquid based on a low-temperature plasma technology is provided, including the following steps:
(1) preparation of surimi: collecting fish flesh, and chopping the fish flesh to obtain the surimi;
(2) preparation of the surimi-rinsing waste liquid: adding the surimi to water, mixing for rinsing, and centrifuging to collect a supernatant, which is the surimi-rinsing liquid;
(3) low-temperature plasma treatment of the surimi-rinsing liquid: adjusting a parameter of a plasma device, including setting an input power to 300 W to 800 W; placing a spray gun of the plasma device below a liquid level of the surimi-rinsing liquid; turning on a power supply; and exciting plasma with air as a gas source, such that the plasma acts evenly on the surimi-rinsing liquid for 2 min to 6 min, to obtain a low-temperature plasma treated surimi-rinsing liquid; and
(4) recovery of the protein: allowing the low-temperature plasma treated surimi-rinsing liquid to stand in an environment with a temperature of 10° C. or lower, and centrifuging to collect a precipitate, such that the recovery of the protein from the surimi-rinsing liquid is completed.

Preferably, in the step (2), a ratio of the surimi to the water is 1 g:5 mL; and the water is distilled water.

Preferably, in the step (2), the rinsing is conducted for 5 min to 10 min.

Preferably, in the step (2), the centrifuging is conducted at 4° C., and 8,000 r/min for 10 min to 15 min.

Preferably, in the step (3), the parameter is the output power of 300 W to 800 W.

Preferably, in the step (3), a height of the surimi-rinsing liquid is 15 cm to 20 cm.

Preferably, in the step (3), the spray gun is fixed at a position 10 mm to 15 mm below the liquid level of the surimi-rinsing liquid.

Preferably, in the step (3), the low-temperature plasma treatment is conducted for 4 min.

Preferably, in the step (4), the low-temperature plasma treated surimi-rinsing liquid is allowed to stand at 0° C. to 4° C. for 6 h.

Preferably, in the step (4), the centrifuging is conducted at 4° C., and 8,000 r/min for 10 min to 15 min.

Determination of a protein recovery rate: A supernatant obtained after the centrifugation in step (4) is placed in a 20° C. water bath, allowed to stand for 90 min, and then centrifuged at 4,000 r/min for 10 min, and a final supernatant is collected for protein concentration determination; and finally, a protein concentration in the original surimi-rinsing liquid and a protein concentration in the final supernatant are determined by a biuret method, and the protein recovery rate is calculated as follows:

$$Protein recovery rate ( \% ) = ( 1 - \frac{{C}_{1}}{{C}_{0}} ) \times 100$$

where C₀ represents the protein concentration in the original surimi-rinsing liquid, mg/mL; and C₁ represents the protein concentration in the final supernatant, mg/mL.

The protein recovery rate is used to evaluate an effect of a low-temperature plasma treatment to recover the protein from a surimi-rinsing liquid.

### Beneficial Effects

In the present disclosure, a prepared surimi-rinsing liquid is treated with a low-temperature plasma technology to precipitate and recover proteins in the surimi-rinsing liquid. Compared with the traditional method for recovering proteins from a surimi-rinsing liquid, the direct treatment of a surimi-rinsing liquid by a low-temperature plasma device increases a recovery rate of proteins in the surimi-rinsing liquid, shortens a process flow of protein recovery, improves the recovery efficiency of proteins (a highest protein recovery rate of the low-temperature plasma treatment for a surimi-rinsing liquid is 88.23%), avoids secondary contamination during recovery, and is eco-friendly. The present disclosure provides a feasible method for recovering by-products in industrial production of surimi, thereby further achieving the high-value production of surimi.

The low-temperature plasma treatment can effectively improve a recovery rate of proteins in a surimi-rinsing liquid, but a time of the low-temperature plasma treatment is critical. When a time of the low-temperature plasma treatment is extended from 2 min to 4 min, a protein recovery rate increases from 79.63% to 88.23%, with an increase of 8.6%. In this case, after a surimi-rinsing liquid undergoes a low-temperature plasma treatment, active ingredients will be produced in the surimi-rinsing liquid to oxidize proteins in the surimi-rinsing liquid, such that a protein structure is expanded and hydrophobic amino acids on a surface of a protein are exposed to make the surface hydrophobicity of the protein change. With the increase of low-temperature plasma treatment time, the active components produced during low-temperature plasma treatment enhanced the adhesion of water micelles to protein molecules, thereby enhancing the surface hydrophobicity of proteins. Low-temperature plasma treatment is beneficial to induction of protein-protein interactions and promotion of the production of protein aggregates, thereby improving protein precipitation and recovery. When a time of the low-temperature plasma treatment is extended from 4 min to 6 min, a protein recovery rate decreases by 9.97%. In this case, the further extension of a time of the low-temperature plasma treatment for a surimi-rinsing liquid causes excessive oxidation of proteins in the surimi-rinsing liquid, which negatively affects the aggregation stability of proteins and makes protein aggregates ruptured, thereby reducing a precipitation and recovery rate of proteins.

### DESCRIPTION OF THE EMBODIMENTS

A plurality of exemplary embodiments of the present disclosure are now described in detail. The detailed description should not be considered as a limitation to the present disclosure, but should be understood as a more detailed description of some aspects, features, and implement solutions of the present disclosure.

It should be understood that terms described in the present disclosure are merely used to describe specific embodiments and are not intended to limit the present disclosure. In addition, for a numerical range in the present disclosure, it should be understood that each intermediate value between an upper limit and a lower limit of the range is also specifically disclosed. Each small range between any stated value or an intermediate value in a stated range and any other stated value or an intermediate value in the stated range is also included in the present disclosure. Upper and lower limits of each of these small ranges can independently be included in or excluded from the range.

Unless otherwise stated, all technical and scientific terms used herein have the same meaning as commonly understood by those of ordinary skill in the art described in the present disclosure. Although only preferred methods and materials are described in the present disclosure, any methods and materials similar or equivalent to those described herein can also be used in the practice or testing of the present disclosure. All documents mentioned in this specification are incorporated by reference to disclose and describe methods and/or materials related to the documents. In case of conflict with any incorporated documents, the content of this specification shall prevail.

It is obvious to a person skilled in the art that a plurality of modifications and variations can be made to the specific embodiments of the present specification without departing from the scope or spirit of the present disclosure. Other embodiments derived from the description of the present disclosure will be obvious to the skilled person. The specification and examples of the present disclosure are merely exemplary.

A raw material used for preparing surimi in the present disclosure is bighead carp, and is purchased from the Zhenjiang RT-Mart Supermarket.

### Example 1

The method of the present disclosure includes steps such as preparation of a surimi-rinsing liquid, low-temperature plasma treatment of the surimi-rinsing liquid, precipitation and recovery of proteins, and data processing. A specific production process is as follows:

A method for recovering a protein from a surimi-rinsing liquid based on a low-temperature plasma technology was provided, including the following steps:

(1) Preparation of surimi: Bighead carp was stunned, the fish skin was removed, and boneless fish flesh was collected manually and chopped by a chopper for 3 min to obtain the surimi.

(2) Preparation of the surimi-rinsing liquid: The surimi was mixed with distilled water at 1:5 (w/v, g/mL) for 5 min to allow rinsing, and the resulting system was centrifuged at 4° C., and 8.000 r/min for 15 min to collect a supernatant, which was the surimi-rinsing liquid.

(3) Low-temperature plasma treatment of the surimi-rinsing liquid: A PG-1000ZD low-temperature plasma torch device was adopted, and an input power of the low-temperature plasma torch device was adjusted to 500 W; the surimi-rinsing liquid was fed into the low-temperature plasma torch device with a spray gun of the device fixed at a position 10 mm below a liquid level of the surimi-rinsing liquid; and a power supply was turned on, and plasma was excited with air as a gas source, such that the plasma acted evenly on the surimi-rinsing liquid for 2 min, to obtain a low-temperature plasma treated surimi-rinsing liquid.

(4) Recovery of the protein: The low-temperature plasma treated surimi-rinsing liquid was allowed to stand at 4° C. for 6 h, and centrifuged at 4° C., and 8,000 r/min for 15 min to collect a precipitate, such that the recovery of the protein from the surimi-rinsing liquid was completed.

(5) A supernatant obtained after the centrifugation in step (4) was placed in a 20° C. water bath, allowed to stand for 90 min, and then centrifuged at 4,000 r/min for 10 min, and a final supernatant was collected for protein concentration determination; and finally, a protein concentration in the original surimi-rinsing liquid and a protein concentration in the final supernatant were determined by a biuret method, and a protein recovery rate was calculated as follows:

$$Protein recovery rate ( \% ) = ( 1 - \frac{{C}_{1}}{{C}_{0}} ) \times 100$$

where C₀ represents the protein concentration in the original surimi-rinsing liquid, mg/mL; and C₁ represents the protein concentration in the final supernatant, mg/mL.

(6) It was determined that a protein recovery rate in this example was 79.63%.

### Example 2

The method of the present disclosure includes steps such as preparation of a surimi-rinsing liquid, low-temperature plasma treatment of the surimi-rinsing liquid, precipitation and recovery of proteins, and data processing. A specific production process is as follows:

A method for recovering a protein from a surimi-rinsing liquid based on a low-temperature plasma technology was provided, including the following steps:

(1) Preparation of surimi: Bighead carp was stunned, the fish skin was removed, and boneless fish flesh was collected manually and chopped by a chopper for 3 min to obtain the surimi.

(2) Preparation of the surimi-rinsing liquid: The surimi was mixed with distilled water at 1:5 (w/v, g/mL) for 5 min to allow rinsing, and the resulting system was centrifuged at 4° C., and 8,000 r/min for 15 min to collect a supernatant, which was the surimi-rinsing liquid.

(3) Low-temperature plasma treatment of the surimi-rinsing liquid: A PG-1000ZD low-temperature plasma torch device was adopted, and an input power of the low-temperature plasma torch device was adjusted to 500 W; the surimi-rinsing liquid was fed into the low-temperature plasma torch device with a spray gun of the device fixed at a position 10 mm below a liquid level of the surimi-rinsing liquid; and a power supply was turned on, and plasma was excited with air as a gas source, such that the plasma acted evenly on the surimi-rinsing liquid for 4 min. to obtain a low-temperature plasma treated surimi-rinsing liquid.

(4) Recovery of the protein: The low-temperature plasma treated surimi-rinsing liquid was allowed to stand at 4° C. for 6 h, and centrifuged at 4° C., and 8.000 r/min for 15 min to collect a precipitate, such that the recovery of the protein from the surimi-rinsing liquid was completed.

(5) A supernatant obtained after the centrifugation in step (4) was placed in a 20° C. water bath, allowed to stand for 90 min, and then centrifuged at 4,000 r/min for 10 min, and a final supernatant was collected for protein concentration determination; and finally, a protein concentration in the original surimi-rinsing liquid and a protein concentration in the final supernatant were determined by a biuret method, and a protein recovery rate was calculated as follows:

$$Protein recovery rate ( \% ) = ( 1 - \frac{{C}_{1}}{{C}_{0}} ) \times 100$$

where C₀ represents the protein concentration in the original surimi-rinsing liquid, mg/mL; and C₁ represents the protein concentration in the final supernatant, mg/mL.

(6) It was determined that a protein recovery rate in this example was 88.23%.

### Example 3

The method of the present disclosure includes steps such as preparation of a surimi-rinsing liquid, low-temperature plasma treatment of the surimi-rinsing liquid, precipitation and recovery of proteins, and data processing. A specific production process is as follows:

A method for recovering a protein from a surimi-rinsing liquid based on a low-temperature plasma technology was provided, including the following steps:

(1) Preparation of surimi: Fresh bighead carp was purchased from a supermarket and stunned, the fish skin was removed, and boneless fish flesh was collected manually and chopped by a chopper for 3 min to obtain the surimi.

(2) Preparation of the surimi-rinsing liquid: The surimi was mixed with distilled water at 1:5 (w/v, g/mL) for 5 min to allow rinsing, and the resulting system was centrifuged at 4° C., and 8.000 r/min for 15 min to collect a supernatant, which was the surimi-rinsing liquid.

(3) Low-temperature plasma treatment of the surimi-rinsing liquid: A PG-1000ZD low-temperature plasma torch device was adopted, and an input power of the low-temperature plasma torch device was adjusted to 500 W; the surimi-rinsing liquid was fed into the low-temperature plasma torch device with a spray gun of the device fixed at a position 10 mm below a liquid level of the surimi-rinsing liquid; and a power supply was turned on, and plasma was excited with air as a gas source, such that the plasma acted evenly on the surimi-rinsing liquid for 6 min, to obtain a low-temperature plasma treated surimi-rinsing liquid.

(4) Recovery of the protein: The low-temperature plasma treated surimi-rinsing liquid was allowed to stand at 4° C. for 6 h, and centrifuged at 4° C., and 8,000 r/min for 15 min to collect a precipitate, such that the recovery of the protein from the surimi-rinsing liquid was completed.

(5) A supernatant obtained after the centrifugation in step (4) was placed in a 20° C. water bath, allowed to stand for 90 min, and then centrifuged at 4,000 r/min for 10 min, and the a final supernatant was collected for protein concentration determination; and finally, a protein concentration in the original surimi-rinsing liquid and a protein concentration in the final supernatant were determined by a biuret method, and a protein recovery rate was calculated as follows:

$$Protein recovery rate ( \% ) = ( 1 - \frac{{C}_{1}}{{C}_{0}} ) \times 100$$

where C₀ represents the protein concentration in the original surimi-rinsing liquid, mg/mL; and C₁ represents the protein concentration in the final supernatant, mg/mL.

(6) It was determined that a protein recovery rate in this example was 78.26%.

Finally, it should be noted that the above examples are merely intended to illustrate the present disclosure, rather than to limit the technical solutions described in the present disclosure. Therefore, although the present disclosure is described in detail in this specification with reference to the above-mentioned examples, those of ordinary skill in the art should understand that the present disclosure can still be modified or equivalently replaced. All technical solutions and improvements thereof made without departing from the spirit and scope of the present disclosure should be covered by the scope of the claims of the present disclosure.

1. A method for recovering a protein from a surimi-rinsing liquid based on a low-temperature plasma technology, comprising the following steps:
(1) collecting fish flesh, and chopping the fish flesh to obtain surimi;
(2) preparation of the surimi-rinsing liquid: adding the surimi to water, mixing for rinsing, and centrifuging to collect a supernatant, which is the surimi-rinsing liquid;
(3) low-temperature plasma treatment of the surimi-rinsing liquid: adjusting a parameter of a plasma device, comprising setting an input power to 300 W to 800 W; placing a spray gun of the plasma device below a liquid level of the surimi-rinsing liquid; turning on a power supply; and exciting plasma with air as a gas source, such that the plasma acts evenly on the surimi-rinsing liquid for 2 min to 6 min, to obtain a low-temperature plasma treated surimi-rinsing liquid; and
(4) recovery of the protein: allowing the low-temperature plasma treated surimi-rinsing liquid to stand in an environment with a temperature of 10° C. or lower, and centrifuging to collect a precipitate, such that the recovery of the protein from the surimi-rinsing liquid is completed.

2. The method for recovering the protein from the surimi-rinsing liquid based on the low-temperature plasma technology according to claim 1, wherein in the step (2), a ratio of the surimi to the water is 1 g:5 mL; and the water is distilled water.

3. The method for recovering the protein from the surimi-rinsing liquid based on the low-temperature plasma technology according to claim 1, wherein in the step (2), the rinsing is conducted for 5 min to 10 min.

4. The method for recovering the protein from the surimi-rinsing liquid based on the low-temperature plasma technology according to claim 1, wherein in the step (2), the centrifuging is conducted at 4° C., and 8,000 r/min for 10 min to 15 min.

5. The method for recovering the protein from the surimi-rinsing liquid based on the low-temperature plasma technology according to claim 1, wherein in the step (3), the parameter is the output power of 300 W to 800 W.

6. The method for recovering the protein from the surimi-rinsing liquid based on the low-temperature plasma technology according to claim 5, wherein in the step (3), a height of the surimi-rinsing liquid is 15 cm to 20 cm.

7. The method for recovering the protein from the surimi-rinsing liquid based on the low-temperature plasma technology according to claim 1, wherein in the step (3), the spray gun is fixed at a position 10 mm to 15 mm below the liquid level of the surimi-rinsing liquid.

8. The method for recovering the protein from the surimi-rinsing liquid based on the low-temperature plasma technology according to claim 1, wherein in the step (3), the low-temperature plasma treatment is conducted for 4 min.

9. The method for recovering the protein from the surimi-rinsing liquid based on the low-temperature plasma technology according to claim 1, wherein in the step (4), the low-temperature plasma treated surimi-rinsing liquid is allowed to stand at 0° C. to 4° C. for 6 h.

10. The method for recovering the protein from the surimi-rinsing liquid based on the low-temperature plasma technology according to claim 1, wherein in the step (4), the centrifuging is conducted at 4° C., and 8,000 r/min for 10 min to 15 min.
